(19) United States
(12) Patent Application Publication
(10) Pub. No.: US 2022/0250061 A1
(43) Pub. Date: Aug. 11, 2022

(54) MULTI-FLUID DENSITY GRADIENT COLUMNS

(71) Applicant: Hewlett-Packard Development Company, L.P., Spring, TX (US)

(72) Inventors: Si-Lam J. Choy, Corvallis, OR (US); Hilary ELY, Corvallis, OR (US)

(73) Assignee: Hewlett-Packard Development Company, L.P., Spring, TX (US)

(21) Appl. No.: 17/630,592

(22) PCT Filed: Oct. 29, 2019
(86) PCT No.: PCT/US2019/058429

§ 371 Date: Jan. 27, 2022

(51) Int. Cl. B01L 3/00; B03C 1/01; B03C 1/28

(52) U.S. Cl. CPC B01L 3/502715; B03C 1/01; B03C 1/288; B03C 2201/18; B01L 2200/0668; B01L 2300/0663; B01L 2200/027

(57) ABSTRACT

The present disclosure includes a method of forming and loading a multi-fluid density gradient column. The method can include forming a multi-fluid density gradient column and loading magnetizing microparticles into a first fluid layer or a second fluid layer of the multi-fluid density gradient column. Forming the multi-fluid density gradient column can include loading a first fluid having a first fluid density in a multi-fluid density gradient column to form a first fluid layer and loading a second fluid having a second fluid density greater than the first fluid density in the multi-fluid density gradient column to form a second fluid layer. The multi-fluid density gradient column can be fluidly coupled to a fluid processing device. The magnetizing microparticles can be surface-activated to bind with a biological component or can be bound to the biological component.

### BACKGROUND

In biomedical, chemical, and environmental testing, isolating a biological component from a sample can be useful. The separation can permit analysis or amplification, for example. As the quantity of available assays for biological components increases, so does the demand for the ability to isolate such components from samples.

### BRIEF DESCRIPTION OF THE DRAWING

FIG. 1 is a flow diagram illustrating an example method of forming and loading a multi-fluid density gradient column in accordance with the present disclosure;

FIG. 2 graphically illustrates a schematic view of an example method of forming and loading a multi-fluid density gradient column in accordance with the present disclosure;

FIG. 3 is a flow diagram illustrating an example method of forming and loading a multi-fluid density gradient column in accordance with the present disclosure;

FIG. 4 graphically illustrates a schematic view of an example microfluidic biological component concentration and processing system in accordance with the present disclosure; and

FIG. 5 graphically illustrates a schematic view of an example microfluidic biological component concentration and processing system in accordance with the present disclosure.

### DETAILED DESCRIPTION

In biological assays, a biological component can be intermixed with other components in a biological sample that can interfere with subsequent analysis. As used herein, the term “biological component” can refer to materials of various types, including proteins, cells, cell nuclei, nucleic acids, bacteria, viruses, and the like, that can be present in a biological sample. A “biological sample” can refer to a sample obtained for analysis from a living or deceased organism. In an example, a biological sample can include blood, sputum, urine, tissues, fecal matter, and the like. Isolating the biological component from other components in a biological sample can permit analysis of the biological component that would not be easy if the biological component remained in the biological sample. However, current isolation techniques can include repeated dispersing and re-aggregating. The repeated dispersing and re-aggregating can result in a loss of a quantity of the biological component. Following isolation, current techniques further include transferring the biological component to a processing device for subsequent analysis, which can further result in a loss of quantity of the biological component and add processing time. Therefore, isolating a biological component from other components in the biological sample and analyzing the biological component using current techniques can be complex, time consuming, and labor intensive and can also result in less than maximum yields of the isolated biological component.

In accordance with examples of the present disclosure, a method of forming and loading a multi-fluid density gradient column includes, for example, forming a multi-fluid density gradient column by loading a first fluid having a first fluid density to form a first fluid layer, and loading a second fluid having a second fluid density greater than the first fluid density to form a second fluid layer. In this example, the multi-fluid density gradient column is fluidly coupled to a fluid processing device. In additional detail, in this example, the method further includes loading magnetizing microparticles that are surface-activated to bind with a biological component, or which are bound to the biological component, into the first fluid layer or the second fluid layer of the multi-fluid density gradient column. In one more specific example, the second fluid can be loaded from a bottom of the multi-fluid density gradient column to form the second fluid layer and the first fluid can be loaded from a top of the multi-fluid density gradient column to form the first fluid layer. In another example, the first fluid and the second fluid can be loaded sequentially from a bottom of the multi-fluid density gradient column to form the first fluid layer positioned on top of the second fluid layer. In another example, the method can include loading a third fluid having a third fluid density in the multi-fluid density gradient column, wherein the third fluid forms a third fluid layer based on the third fluid density in relation to the first fluid density of the first fluid and the second fluid density of the second fluid. The method can also include adjusting the first density of the first fluid, adjusting the second density of the second fluid, or adjusting both the first density of the first fluid and the second density of the second fluid prior to loading the first fluid and the second fluid into the multi-fluid density gradient column so that the second fluid density becomes greater than the first fluid density or so that a difference in the greater density of the second fluid density increases relative to the first fluid density. In one specific example, the multi-fluid density gradient column can include an inverted T-pipe associated with a valve, trapped gas, or a combination thereof to trap the second fluid in a channel extending upward from the inverted T-pipe.

In another example, a method of using a multi-fluid density gradient column in sample analysis, in one example, includes loading a biological sample including a biological component and magnetizing microparticles that are surface-activated to bind with the biological component of the biological sample, or which are bound to the biological component of the biological sample, into a first fluid layer or a second fluid layer of a multi-fluid density gradient column. The first fluid layer in this example includes a first fluid having a first fluid density and the second fluid layer includes a second fluid having a second fluid density greater than the first fluid density. The method further includes, by way of example, exposing the magnetizing microparticles including the biological component bound thereto to a magnetic field to move the magnetizing microparticles including the biological component bound thereto from the first fluid layer into the second fluid layer. In further detail, this example method further includes passing the biological component to a fluid processing device through a fluidic outlet of the multi-fluid density gradient column and analyzing the biological component in the fluid processing device. In one example, the method can include admixing the magnetizing microparticles and the biological sample in a loading solution before loading the biological sample and the magnetizing microparticles into the first fluid layer or the second fluid layer of the multi-fluid density gradient column. Furthermore, the method can include passing of the biological component to the fluid processing device, including pumping, e.g., positive or negative pressure pumping, the biological component into the fluid processing device via an injection pump, a syringe pump, a diaphragm pump, a peristaltic pump, or a combination thereof. Furthermore, the method can include coating exposed surfaces on the magnetizing microparticles including the biological component bound thereto with a blocking agent prior to the analyzing of the biological component in the fluid processing device. The method can also include dissociating the biological component from the magnetizing microparticles prior to the analyzing of the biological component in the fluid processing device. In an example, the fluid processing device can include active circuitry including a sensor selected from a photo sensor, a thermal sensor, an optical sensor, a fluid flow sensor, a chemical sensor, an electrochemical sensor, a MEMS, or a combination thereof.

In another example, a microfluidic biological component concentration and processing system includes magnetizing microparticles that are surface-activated to bind with a biological component, or which are bound to the biological component, and a multi-fluid density gradient column to receive or contain the magnetizing microparticles. The multi-fluid density gradient column in this example includes a first fluid layer having a first fluid density and a second fluid layer having a second fluid density that is greater than the first fluid density of the first fluid, wherein the second fluid layer is positioned vertically beneath the first fluid layer. This system also includes, for example, a magnet to draw the magnetizing microparticles from the first fluid layer into the second fluid layer, a fluidic outlet fluidly coupled to the fluid layer or the second fluid layer, and a fluid processing device to receive modified fluid from the multi-fluid density gradient column, wherein the fluid processing device includes electronic circuitry that is interactive with the modified fluid. In one example, the fluid processing device can include a microfluidic chip with a microfluidic channel. The microfluidic chip can also include active circuitry positioned to interact with the modified fluid, the biological component, or both within the microfluidic channel. In further detail, the processing system can include a first multi-fluid density gradient column associated with a first fluidic outlet that is fluidically connected to a first fluid processing device, and a second multi-fluid density gradient column associated with a second fluidic outlet that is fluidically connected to a second fluid processing device.

It is noted that when discussing methods of loading a multi-fluid density gradient column, methods of using a multi-fluid density gradient column in sample analysis, and microfluidic biological component concentration and processing systems herein, such discussions can be considered applicable to one another whether or not they are explicitly discussed in the context of that example. Thus, for example, when discussing a multi-fluid density gradient column in the method of forming and loading a multi-fluid density gradient column, such disclosure is also relevant to and directly supported in the context of the method of using a multi-fluid density gradient column in sample analysis or the microfluidic biological component concentration and processing system, and vice versa.

Terms used herein will have the ordinary meaning in the relevant technical field unless specified otherwise. In some instances, there are terms defined more specifically throughout the specification or included at the end of the present specification, and thus, these terms can have a meaning as described herein.

### Methods of Loading a Multi-Fluid Density Gradient Columns

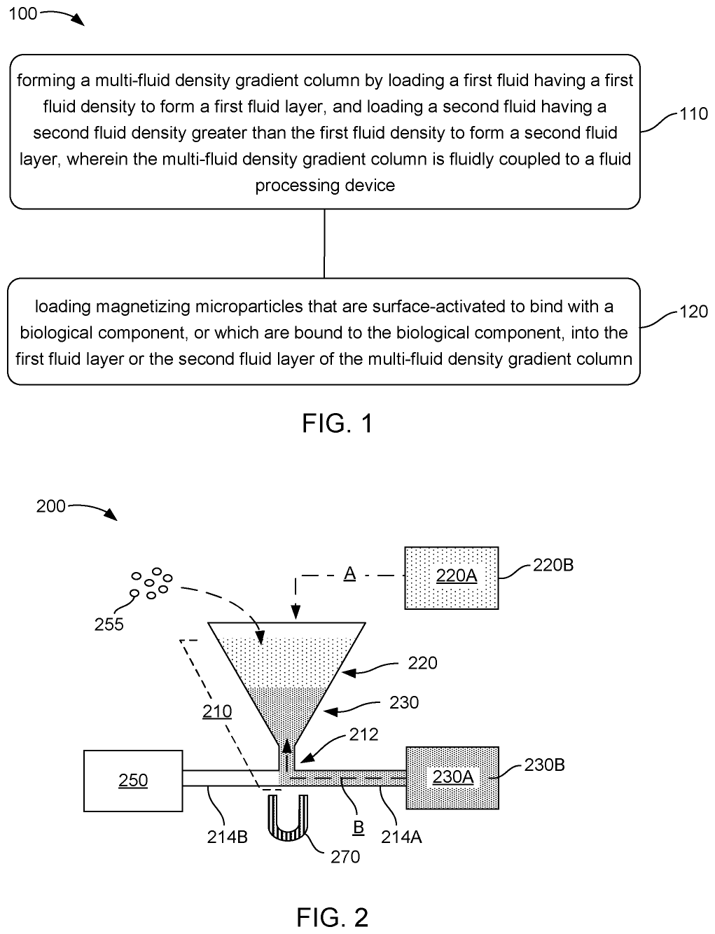

A method 100 of loading a multi-fluid density gradient column is shown in FIG. 1, and can include forming 110 a multi-fluid density gradient by loading a first fluid having a first fluid density in a multi-fluid density gradient column to form a first fluid layer, and loading a second fluid having a second fluid density greater than the first fluid density in the multi-fluid density gradient column to form a second fluid layer, wherein the multi-fluid density gradient is fluidly coupled to a fluid processing device. The method further includes loading 120 magnetizing microparticles that are surface-activated to bind with a biological component, or which are bound to the biological component, into the first fluid layer or the second fluid layer of the multi-fluid density gradient column.

As schematically illustrated in FIG. 2, a system 200 is shown to illustrate methods of forming a multi-fluid density gradient column 210, which can include loading a first fluid 220A that can have a first fluid density, such as from a first fluid vessel 220B, to be included in the multi-fluid density gradient column as a first fluid layer 220, and loading a second fluid 230A that can have a second fluid density greater than the first fluid density, and can be supplied by a second fluid vessel 230B to be included in the multi-fluid density gradient column as a second fluid layer 230. In this example, the first fluid can be top loaded, shown at A, via a top opening of the vessel that contains the multi-fluid density gradient channel, and the second fluid can be bottom loaded, shown at B, via a first fluidic channel 214A and up through fluidic opening 212, which in this instance is an inverted fluidic T-pipe channeler that may also act as a fluidic outlet when releasing fluids from the multi-fluid density gradient column into a second fluidic channel 214B. This inverted T-pipe channeler may be fluidically (e.g., gas or liquid) or mechanically valved. For example, first and second fluidic channels may be pressurized with gas so that the second fluid is forced upwards into the multi-fluid density gradient column through the inverted fluidic T-pipe channeler when the second fluid is pumped, drawn, or otherwise moved away from the fluid source through the second fluidic channel. In this arrangement, the first fluid can be loaded first, the second fluid can be loaded first, or the first fluid and the second fluid can be loaded simultaneously.

Following the formation of the multi-fluid density gradient column 210, or as part of introducing the various fluids into the multi-fluid density gradient column, the magnetizing microparticles 255 can be loaded into the first fluid layer 220 (as shown) or the second fluid layer (not shown), or the magnetizing microparticles can be preloaded in the first fluid or the second fluid (or other fluid layer that may also be present). The magnetizing microparticles can be surface-activated to bind with a biological component or can be bound to the biological component in preparation for introduction into the multi-fluid density gradient column. With the magnetizing microparticles loaded in the multi-fluid density gradient column, a magnet 270 can be used to draw the magnetizing microparticles from the first fluid layer into the second fluid layer, and in some instances, further through the fluidic opening 212 (or inverted fluidic T-pipe channeler) to be channeled through second fluidic channel 214B and into a fluid processing device 250, for example. In other examples, the fluid processing device may be directly beneath the multi-fluid density gradient column and the fluid processing device can be loaded directly without intervening fluidics. In other examples, the fluid processing device can be considered to be a downstream fluid processing device, meaning that the fluid processing device is positioned downstream from the multi-fluid density gradient column, either through intervening fluidic channels or directly coupled to the multi-fluid gradient density column. In still other examples, the multi-fluid density gradient column may be housed or partially housed by the fluid processing device. Thus, the fluid processing device may be a downstream fluid processing device, or it may integrated as part (or all) of the housing that contains the multi-fluid density gradient column fluid layers.

The fluid processing device can include any of a number of chambers, channels, electronic components, thermocyclers, or other processing components. The fluid processing device can be any configuration that is suitable for performing a function. For example, the fluid processing device can include a chamber or channel to receive the biological component or the magnetizing microparticles with the biological component bound thereto from the multi-fluid density gradient column. The fluid processing device can include a microfluidic chamber, a microfluidic channel, a microfluidic chip, or the like. In one example, the fluid processing device can include a thermal reaction chamber for amplification and detection, such as PCR or LAMP.

In some specific examples, the fluid processing device can include or be made of a material such as metal, glass, silicon, silicon dioxide, a ceramic material (e.g., alumina, aluminum borosilicate, etc.), a polymer material (e.g., polyethylene, polypropylene, polycarbonate, poly(methyl methacrylate), epoxy molding compound, polyamide, liquid crystal polymer (LCP), polyphenylene sulfide, etc.), the like, or a combination thereof. Any of a number of structural designs may be used, including channeling within support substrates, combinations of support substrates and lids that are architecturally compatible to form a seal or receive a sealing material at their interface, etc.

In other examples, the fluid processing device can also include a pumping component such as an injection pump, a syringe pump, a diaphragm pump, a peristaltic pump, or a combination thereof. The pumping component may be a suctioning component, for example, to pull the biological component, and in some examples a fluid layer such as a master mix layer, into the fluid processing device and/or through the fluid processing device.

In yet other examples, the fluid processing device can include active circuitry, such as fluid actionable circuitry and/or transistor circuitry. “Active circuitry” is defined as electronic circuitry that can be electrically operated to interact (or cause interactions) with a biological component or fluid carrying a biological component within a fluid processing device. The interactions that result may be electrical, mechanical, optical, and/or chemical, for example. For example, active circuitry can include components that can operate as a heater (e.g., rapid thermal cycling heater, resistive heater, etc.), a sensor (e.g., photo sensor, thermal sensor, optical sensor, fluid flow sensor, chemical sensor, electrochemical sensor, MEMS, etc.), an electromagnetic radiation source (e.g., LED or other photo diode, laser, etc.), a fluid actuator (e.g., mixers, bubblers, pumps, etc.), or the like. Within a fluid processing device, there can be a single active circuitry component, an array or one type of active circuitry components, multiple types of active circuitry circuits, arrays of multiple types of active circuitry circuits, or any combination thereof. The active circuitry can be positioned to physically contact a fluid when fluid including a biological component is introduced into the fluid processing device, or there may be a thin protective film or layer of material that protects the active circuitry from the fluid, but which does not interfere the function of the active circuitry in interacting with a fluid or target substance of a fluid. For example, there may be a protective film(s) or layer(s) of polymer, oxide, carbide, metal or alloy, nitride, silicon, etc. The film(s) or layer(s) thickness can be thin enough that the active circuitry can interact with the fluid or the target substance therein. In some examples, active circuity can protrude into the microfluidic chamber or channel configured to hold a fluid with a biological component therein. Circuitry can include, for examples, capacitors, resistors, inductors, transistors, amplifiers, diodes, e.g., LEDs, integrated circuits, etc. In one example, there may be transistor circuitry, which may be electrically configured to provide onboard logic to the fluid processing device.

In an example, the fluid processing device can include a reaction chamber, optical sensor, electrochemical sensor, heater, pump, input port, outlet port, or a combination thereof. In another example, the fluid processing device may include multiple reaction chambers in parallel or in series. In an example, the fluid processing device can be included as part of a lab-on-a-chip device.

The term “multi-fluid density gradient column” as used herein, can refer to a multi-layered fluid column where individual fluid layers are separated from one another based on phase separation and density differentials from layer to layer. There can be any of a number of multiple layers, such as from 2 fluid layers to 12 fluid layers, from 2 fluid layers to 8 fluid layers, from 3 fluid layers to 12 fluid layers, from 3 fluid layers to 8 fluid layers, from 3 fluid layers to 6 fluid layers, from 2 fluid layers to 4 fluid layers, or from 3 fluid layers to 5 fluid layers, for example. A multi-fluid density gradient column is formed with multiple fluid layers phase separated from one another without a physical barrier therebetween. If there are physical barriers therebetween, there are still some pairs of layers that are phase separated with densities different enough to remain phase separated. Fluids with larger fluid density values relative to other fluids become located beneath fluids with smaller fluid density values.

As used herein, numerical indicators such as “first” and “second” are not intended to denote loading order. These terms are utilized to distinguish a portion of one fluid in the multi-fluid density gradient column from another portion of another fluid in the multi-fluid density gradient column. Order of loading is not determined by these numerical distinguishers. In addition, loading from the bottom or the top of the multi-fluid density gradient column does not indicate loading in sequential order of the fluid layers of the multi-fluid density gradient column as the fluid layers can self-arrange based on density.

In one example, the first fluid can be loaded before the second fluid can be loaded into the multi-fluid density gradient column. In another example, the second fluid can be loaded before the first fluid can be loaded into the multi-fluid density gradient column. In yet another example, the second fluid can be loaded from a bottom of the multi-fluid density gradient column to form the second fluid layer and the first fluid can be loaded from a top of the multi-fluid density gradient column to form the first fluid layer. In a further example, the first fluid and the second fluid can be loaded sequentially from a bottom of the multi-fluid density gradient column to form the first fluid layer positioned on top of the second fluid layer.

A quantity of fluid layers in the multi-fluid density gradient column is not particularly limited. In one example, the method can further include loading a third fluid having a third fluid density in the multi-fluid density gradient column. The third fluid can form a third fluid layer based on the third fluid density in relation to the first fluid density of the first fluid and the second fluid density of the second fluid. In further examples, the method can further include loading a fourth, fifth, or sixth fluid to the multi-fluid density gradient column. The fourth, fifth, or sixth fluid can be phase separated from other fluids in the multi-fluid density gradient column based on a density of the fourth, fifth, or sixth fluid with respect to the other fluids in the multi-fluid density gradient column.

As previously asserted, maintaining a sequential arrangement of the fluid layers can occur based on fluid density. In some examples, the method can further include adjusting a density of a fluid before loading the fluid into the multi-density gradient column. For example, the method can include adjusting the first density of the first fluid, adjusting the second density of the second fluid, or adjusting both the first density of the first fluid and the second density of the second fluid prior to loading the first fluid and the second fluid into the multi-fluid density gradient column. In one example, adjusting a fluid density can allow a second fluid density to become greater than a first fluid density, so that a difference in the greater density of the second fluid density increases relative to the first fluid density. Increasing the second fluid density to a density greater than a first fluid density of the first fluid can allow the second fluid to be phase separated from and positioned beneath the first fluid in the multi-fluid density gradient column.

Adjusting a fluid density of a fluid can occur by adding a densifier to the fluid. Example densifiers can include sucrose, polysaccharides such as FICOLL™ (commercially available from Millipore Sigma (USA)), C₁₉H₂₆I₃N₃O₉ such as NYCODENZ® (commercially available from Progen Biotechnik GmbH (Germany)) or HISTODENZ™, iodixanols such as OPTIPREP™ (both commercially available from Millipore Sigma (USA)), or combinations thereof. As an amount of densifier in the fluid increases, a density of the fluid can also increase. In further detail, example additives that can be included in the first fluid layer, or in other fluid layers, depending on the design of the multi-fluid gradient column may include sucrose, heat eluted sucrose, C1-C4 alcohol, e.g., isopropyl alcohol, ethanol, etc., which can be included to adjust density, and/or to provide a function with respect to biological component or materials to pass through the column.

In some examples, the method can also include controlling a vertical height of the fluid layers in the multi-fluid density gradient column. A vertical height of the fluid layer can contribute to a residence time of the magnetizing microparticles in the layer. The taller the fluid layer, the longer the residence time of the magnetizing microparticles in the fluid layer. In some examples, the fluid layers in the multi-fluid density gradient column can be the same vertical height. While in other examples, a vertical height of individual fluid layers in a multi-fluid density gradient column can vary from one layer to the next in order to permit a longer residence time in some fluids. In one example, a vertical height of the fluid layers along the multi-fluid density gradient column can individually range from 10 μm to 50 mm. In another example, a vertical height of the fluid layers along the multi-fluid density gradient column can individually range from 10 μm to 30 mm, from 25 μm to 1 mm, from 200 μm to 800 μm, or from 1 mm to 50 mm.

The fluid layers in the multi-fluid density gradient column can be formulated and fluidically assembled so that the surface of the magnetizing microparticles (which may include a biological component attached or attracted thereto) interacts with the different fluids as the magnetizing microparticles pass from layer to layer. Biological material that may be added can include whole blood, platelets, cells, lysed cells, cellular components, nucleic acids, e.g., DNA, RNA, primers, etc., oligo or poly-bases, peptides, or the like. In one example, a fluid layer can include a lysis buffer to lyse cells. In yet other examples, a fluid layer can be a surface binding fluid layer to bind the biological component to the magnetizing microparticles, a wash fluid layer to trap contaminates from a sample fluid and/or remove contaminates from an exterior surface of the magnetizing microparticles, a surfactant fluid layer to coat the magnetizing microparticles, a dye fluid layer, an elution fluid layer to remove the biological component from the magnetizing microparticles following extraction from the biological sample, a labeling fluid layer for binding labels to the biological component such as a fluorescent label (either attached to the magnetizing microparticles or unbound thereto), a reagent fluid layer to prep a biological component for further analysis such as a master mix fluid layer to prep a biological component for PCR, and so on.

In some examples, individual fluid layers can provide sequential processing of a biological component from a biological sample. For example, individual fluid layers can carry out individual functions, and in many cases, the functions can be coordinated to achieve a specific result. For example, in considering biological material found in a cell, sequential fluid layers from top to bottom of a multi-fluid density gradient column can act on the cell to lyse the cell in a first fluid layer, and bind a target biological material from the lysed cell to magnetizing microparticles in a second fluid layer (or lysing and binding can alternatively be done in a single fluid). Additional fluid layers may be used to wash the magnetizing microparticles with the biological material bound thereto in a third fluid layer, e.g., washing the second fluid layer from the magnetizing microparticles in the third fluid layer, and/or elute (or separate) the biological material from the magnetizing microparticles in the fourth fluid. The surface binding and cell lysis can occur, for example, with a lysate buffer in a sucrose and water solution. Washing can occur in a sucrose in water solution, for example. In other examples, individual fluids can be present as a fluid layer(s) along the multi-fluid density gradient column in the form of a master mix fluid for nucleic acid processing. Other combinations of fluid layers (first, second, third, etc.) may include a surfacing binding fluid, a washing fluid, and an elution fluid; or may include a lysis fluid, a washing fluid, a surface binding fluid, a second washing fluid, an elution fluid, and a reagent fluid. Regardless of the various functions of the various fluid layers with sequentially increasing densities arranged from top to bottom, at the individual fluid layers, the magnetizing microparticles can interact with the fluid layer, and may be modified or otherwise changed in some manner based on the function provided by the fluid layer. A series of fluid layers can thus sequentially process the magnetizing microparticles with surface active groups and/or biological material associated therewith or associated with individual fluid layers, for example.

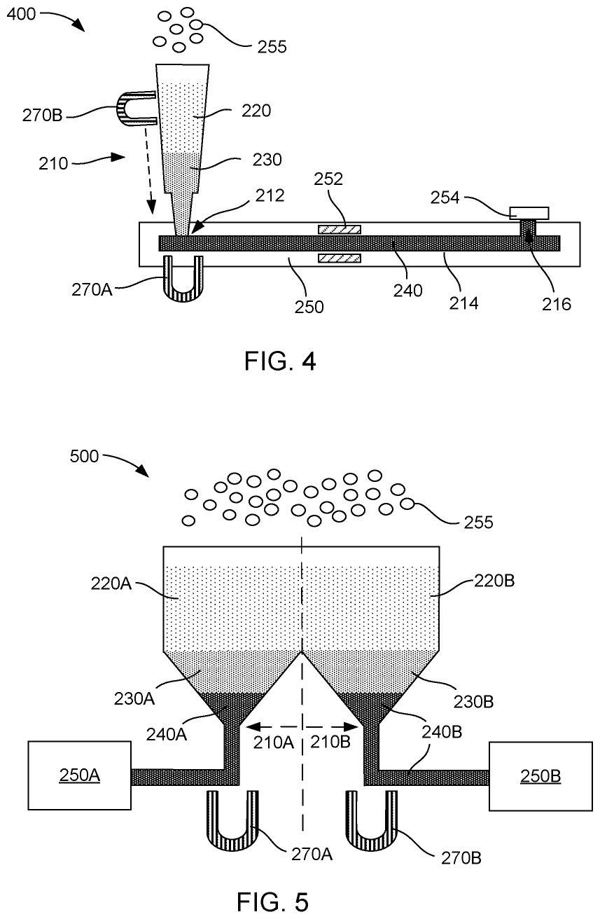

A configuration of the multi-fluid density gradient column, that the fluid layers can be vertically disposed therein, is not particularly limited. However, in one example, the multi-fluid density gradient column can incorporate a fluidic opening 212 fluidly coupling various fluids of the multi-fluid density gradient column, e.g., the inverted fluidic T-pipe channeler shown in FIGS. 2 and 5, which may be used with a mechanical valve, a fluidic valve, e.g., using trapped gas or liquid, or a combination to force fluid up through the opening for loading. Alternatively, the fluidic opening may be present at a fluidic junction joining multiple channeling structures or vessels, as shown at 212 in FIG. 4.

### Methods of Using a Multi-Fluid Density Gradient Column in Sample Analysis

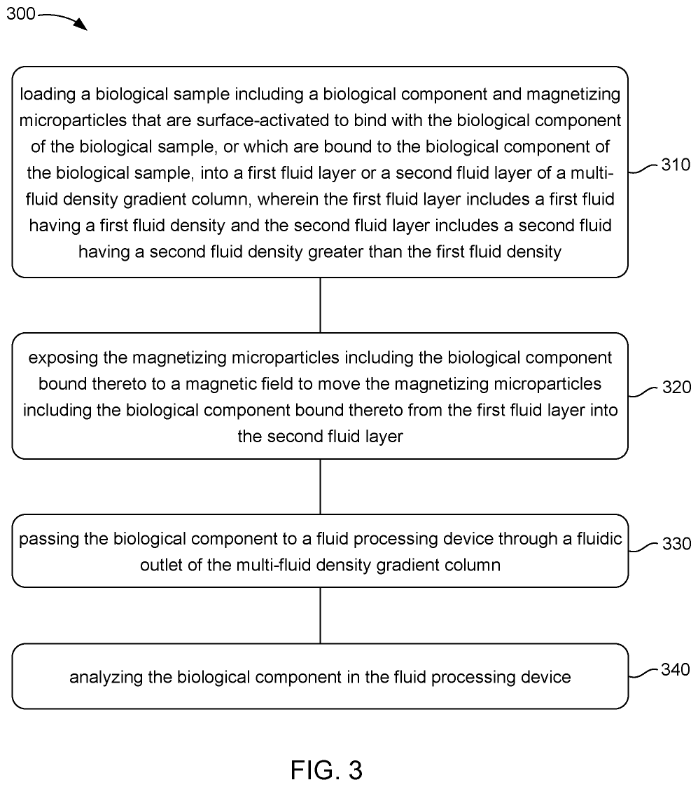

An example flow diagram of a method 300 of using a multi-fluid density gradient column for sample processing or analysis is illustrated in FIG. 3. The method can include loading 310 a biological fluid sample including a biological component and magnetizing microparticles that can be surface-activated to bind with the biological component of the biological sample, or which can be bound to the biological component of the biological sample, into a first fluid layer or a second fluid layer of a multi-fluid density gradient column. The first fluid layer can include a first fluid that can have a first fluid density and the second fluid layer can include a second fluid that can have a second fluid density greater than the first fluid density. The method can further include exposing 320 the magnetizing microparticles including the biological component bound thereto to a magnetic field to move the magnetizing microparticles including the biological component bound thereto from the first fluid layer into the second fluid layer; passing 330 the biological component to a fluid processing device through a fluidic outlet of the multi-fluid density gradient column; and analyzing 340 the biological component in the fluid processing device.

In some examples, the method can further include admixing the magnetizing microparticles and the biological sample in a loading solution prior to loading the biological sample including the biological component and the magnetizing microparticles into the first fluid layer or the second fluid layer of the multi-fluid density gradient column. The loading fluid can include secondary components selected from enzymes, cellular debris, lysing agents, buffers, or a combination thereof. The biological sample and the magnetizing microparticles can be permitted to incubate in the loading fluid for from 30 seconds to 30 minutes or from 2 minutes to 5 minutes. The magnetizing microparticles can be bound to the biological component in a loading fluid prior to loading the magnetizing microparticles and the biological component into the first fluid layer or the second fluid layer of a multi-fluid density gradient column.

In another example, the method can further include coating exposed surfaces on the magnetizing microparticles including the biological component bound thereto with a blocking agent prior to the analyzing of the biological component in the fluid processing device. The blocking agent can coat any remaining exposed surfaces of the magnetizing microparticles and can prevent an interference reaction by the magnetizing microparticles during analysis. A blocking agent can include proteins such as bovine serum albumin and/or casein, enzymes, and/or surfactants such as polysorbate 20, TRITON™-X 100, PLURONIC® F127, and/or PLURONIC® F68 (all available from Millipore Sigma (USA)),or the like, for example. A blocking agent can be included in a fluid layer of the multi-fluid density gradient column.

In further examples, the method can include dissociating the biological component from the magnetizing microparticles prior to the analyzing of the biological component in the fluid processing device. The dissociating can occur by contacting the magnetizing microparticles with an elution fluid. An example elution fluid can include Tris-EDTA, phosphate buffered saline, master mix, or a combination thereof. In an example, an elution fluid can be included in a fluid layer of the multi-fluid density gradient column or can be passed over the magnetizing microparticles after they exit the multi-fluid density gradient column. In yet other examples, the magnetizing microparticles can be heated in a buffer solution to facilitate dissociation of the biological component.

In yet another example, when the multi-fluid density gradient column incorporates an inverted T-pipe, the method can further include opening the valve and/or removing the trapped gas, to allow the biological component, either bound or unbound from the magnetizing microparticles, to pass from the multi-fluid density gradient column to the fluid processing device.

In another example, the method can further include passing of the biological component to the fluid processing device, which can include suctioning the biological component into the fluid processing device via an injection pump, a syringe pump, a diaphragm pump, a peristaltic pump, or a combination thereof. The pump can be included in the fluid processing device or can be a separate pump. The passing of the biological component to the fluid processing device can also include pulling the magnetizing microparticles with the biological component bound thereto into the fluid processing device via the magnet.

The isolation of a biological component from a fluid sample can occur in 1 minute to 7 minutes. During the bulk of that time, there can be binding between the magnetizing microparticles and the biological component. The magnetizing microparticles can pass through the multi-fluid density gradient column in 15 seconds to 120 seconds. In an example analysis, thermo-cycling for PCR, can occur within 5 minutes. The isolation and analysis of the biological component can occur within 15 minutes or less. In yet other examples, the isolation and analysis of the biological can occur within 5 minutes to 15 minutes, within 5 minutes to 12 minutes, or within 10 minutes to 15 minutes.

### Microfluidic Biological Component Concentrations and Processing Systems

In accordance with the present disclosure, various example microfluidic biological component concentration and processing systems 400 are shown in various configurations, including the system shown in FIG. 2 previously, which is described in the context of example methods of loading a multi-fluid density gradient column. In further detail regarding additional systems, FIGS. 4 and 5 illustrate two additional microfluidic biological component concentration and processing systems that can be implemented in accordance with the present disclosure. It is noted that the system shown in FIG. 2 and the example systems shown hereinafter in FIG. 4 and FIG. 5 are specific arrangements that are not intended to be limiting, but rather show example arrangements that can be implemented in accordance with the present disclosure.

Specifically, in FIG. 4, a microfluidic biological component concentration and processing system 400 can include some of the same components previously described with reference to FIG. 2. More specifically, the system can include a multi-fluid density gradient column 210 having a first fluid layer 220 that can include a first fluid with a first fluid density, a second fluid layer 230 that can include a second fluid with a second fluid density greater than the first fluid density. In this specific example the multi-fluid density gradient column can also include a third fluid layer 240 that can include a third fluid having a third fluid density that is greater than the second fluid density. In this example, the third fluid layer is located within a fluid processing device 250, and the second fluid layer and the third fluid layer are in fluid communication along a fluid interface at about the location of fluidic opening 212, which in this instance is a fluidic junction that joins two structures together and allows for fluid communication therebetween. With respect to this particular fluid processing device, there is a fluidic channel 214 to transfer the third fluid of the third fluid layer laterally such as to an egress opening 216 that may be associated with a secondary fluidics component 254, e.g., a cap, a filter, electronic circuitry, a sensor, or a fluid processing chamber, for example. Also shown in this example, the fluid processing device may include electronic circuitry 252 that may be interactive with the third fluid contained therein, which may be in the form of a modified fluid after concentrated biological material from the first fluid layer and/or the second fluid layer is introduced into the third fluid layer. The magnetizing microparticles can be drawn into the third fluid layer, for example, and the concentrated biological material can be eluted therefrom, and then pumped or otherwise moved along fluidic channel 214 to be assay by the electronic components present and in position to interact with the modified fluid, for example.

In further detail, the microfluidic biological component concentration and processing system 400 can include magnetizing microparticles 255 that can be surface-activated to bind with a biological component or can be preloaded with a surface attached or adsorbed biological component. The magnetizing microparticles can move from the first fluid layer 220, to the second fluid layer 230, and into the third fluid layer 240 through a fluidic opening 212 under the influence of a magnet(s). In this example, a first magnet 270A is positioned in alignment beneath the multi-fluid density gradient column to draw the magnetizing microparticles downward. However, additionally or alternatively, a second magnet 270B is positioned along a side of the multi-fluid density gradient column and can move in a downward direction to cause the magnetizing microparticles to move in a downward direction from one fluid to the next with increasing fluid density.

With more specific reference to FIG. 5, this specific microfluidic biological component concentration and processing system 500 can permit analysis of multiple biological components from a single biological fluid, for example. In one example, the multi-fluid density gradient column is bifurcated as a first multi-fluid density gradient column 210A and a second multi-fluid density gradient column 210B. Both multi-fluid density gradient columns share a common first fluid layer 220, and have separate second fluid layers 230A, 230B and third fluid layers 240A, 240B. First magnet 270A and second magnet 270B are positioned individually with respect to the two multi-fluid density gradient columns. Additionally, the first multi-fluid density gradient column can be arranged to feed a first fluid processing device 250A, and the second multi-fluid density gradient column can be arranged to feed a second fluid processing device 250B. In this example, magnetizing microparticles 255 can be pulled through the multi-fluid density gradient columns via multiple magnets 270A, 270B individually associated with the two sides of the multi-fluid density gradient columns.

### Magnetizing Microparticles and Magnetics for Introducing Magnetic Fields

The magnetizing microparticles in the systems and methods described herein can be in the form of paramagnetic microparticles, superparamagnetic microparticles, diamagnetic microparticles, or a combination thereof, for example. The magnetizing microparticles can likewise be surface-activated to bind with a biological component or can be bound to the biological component. The term “magnetizing microparticles” is defined herein to include microparticles that may not be magnetic in nature unless and until a magnetic field is introduced at a strength and proximity to cause them to become magnetic. Their magnetic strength can be dependent on the magnetic field applied and may get stronger as the magnetic flied is increased, or the magnetizing microparticles get closer to the magnetic source that is applying the magnetic field.

In more specific detail, “paramagnetic microparticles” have these properties, in that they have the ability to increase in magnetism when a magnetic field is present; however, paramagnetic microparticles are not magnetic when a magnetic field is not present. In some examples, the paramagnetic microparticles can exhibit no residual magnetism once the magnetic field is removed. A strength of magnetism of the paramagnetic microparticles can depend on the strength of the magnetic field, the distance between a source of the magnetic field and the paramagnetic microparticles, and a size of the paramagnetic microparticles. As a strength of the magnetic field increases and/or a size of the paramagnetic microparticles increases, the strength of the magnetism of the paramagnetic microparticles increases. As a distance between a source of the magnetic field and the paramagnetic microparticles increases the strength of the magnetism of the paramagnetic microparticles decreases. “Superparamagentic microparticles” can act similar to paramagnetic microparticles; however, they can exhibit magnetic susceptibility to a greater extent than paramagnetic microparticles in that the time it takes to become magnetized appears to be near zero seconds. “Diamagnetic microparticles,” on the other hand, can display magnetism due to a change in the orbital motion of electrons in the presence of a magnetic field.

An exterior of the magnetizing microparticles can be surface-activated with surface groups that are interactive with a biological component of a biological sample or can include a covalently attached ligand attached to a surface of the microparticles to likewise bind with a biological component of a biological sample. In some examples, the ligand can include proteins, antibodies, antigens, nucleic acid primers, amino groups, carboxyl groups, epoxy groups, tosyl groups, sulphydryl groups, or the like. The ligand can be selected to correspond with and bind with the biological component and can vary based on the type of biological component being isolated from the biological sample. For example, the ligand can include a nucleic acid primer when isolating a biological component that includes a nucleic acid sequence. In another example, the ligand can include an antibody when isolating a biological component that includes antigen. Commercially available examples of magnetizing microparticles that are surface-activated include those sold under the trade name DYNABEADS® (available from ThermoFischer Scientific (USA)).

In some examples, the magnetizing microparticles can have an average particle size that can range from 0.1 μm to 70 μm. The term “average particle size” describes a diameter or average diameter, which may vary, depending upon the morphology of the individual particle. A shape of the magnetizing microparticles can be spherical, irregular spherical, rounded, semi-rounded, discoidal, angular, sub-angular, cubic, cylindrical, or any combination thereof. In one example, the particles can include spherical particles, irregular spherical particles, or rounded particles. The shape of the magnetizing microparticles can be spherical and uniform, which can be defined herein as spherical or near-spherical, e.g., having a sphericity of >0.84. Thus, any individual particles having a sphericity of <0.84 are considered non-spherical (irregularly shaped). The particle size of the substantially spherical particle may be provided by its diameter, and the particle size of a non-spherical particle may be provided by its average diameter (e.g., the average of multiple dimensions across the particle) or by an effective diameter, e.g., the diameter of a sphere with the same mass and density as the non-spherical particle. In further examples, the average particle size of the magnetizing microparticles can range from 1 μm to 50 μm, from 5 μm to 25 μm, from 0.1 μm to 30 μm, from 40 μm to 60 μm, or from 25 μm to 50 μm.

In an example, the magnet can be capable of generating a magnetic field, such as a magnetic field that can be turned on and off by introducing electrical current/voltage to the magnet. Alternatively, the magnet can be a permanent magnet that can be placed in proximity to the multi-fluid density gradient column to effect the movement of the magnetizing microparticles. The magnet can be permanently placed within this proximity, can be movable along the multi-fluid density gradient column, or can be movable in position and/or out of position to effect movement of the magnetizing microparticles. The magnetizing microparticles can be magnetized by the magnetic field generated by the magnet. In addition, the magnet can create a force capable of pulling the magnetizing microparticles through the multi-fluid density gradient column. When the magnet is turned off or not in appropriate proximity, the magnetizing microparticles can reside in a fluid layer until gravity pulls the magnetizing microparticles through fluid layers of the multi-fluid density gradient column, or they may remain suspended in the fluid layer in which they may reside until the magnetic field is applied thereto. The rate at which gravity pulls the magnetizing microparticles through fluid layers (or leave the magnetizing microparticles within a fluid layer) can be based on a mass of the magnetizing microparticles in combination with a surface tension between fluid layers. The magnet can cause the magnetizing microparticles to move from one fluid layer to another or increase a rate at which the magnetizing microparticles pass from one fluid layer into another.

In an example, the magnet can be positioned below the multi-fluid density gradient column and/or below the fluid processing device and can be in a fixed position or can be moveable in position, out of position, or at variable positions to effect downward movement, rate of movement, or to promote little to no movement of the magnetizing microparticles. In another example, the magnet can be positioned adjacent to a side of the multi-fluid density gradient column and can move vertically to cause the magnetizing microparticles to move therewith. In some examples, the magnet can be a ring magnet. A movable magnet(s) can likewise be positioned adjacent to a side of the multi-fluid density gradient column that is not a ring shape but can be any shape effective for moving magnetizing microparticles along the multi-fluid density gradient column. In some examples, the magnet can be moved along a side and/or along a bottom of the multi-fluid density gradient column to pull the magnetizing microparticles in one direction or another. In one example, the magnet can be used to pull the magnetizing microparticles downwardly through fluid layers of the multi-fluid density gradient column. In yet other examples, the magnet can be used to concentrate the magnetizing microparticles near a side wall of the multi-fluid density gradient column to be moved downward by a movable magnet, or by a magnet positioned beneath the multi-fluid density gradient column. In one example, a magnet used to move magnetizing microparticles downward can be used to reverse the direction of the magnetizing microparticles and can cause the magnetizing microparticles to re-enter a fluid layer that the magnetizing microparticles have previously passed through.

A strength of the magnetic field and the location of the magnet in relation to the magnetizing microparticles can affect a rate at which the magnetizing microparticles move downwardly through the multi-fluid density gradient column and into the fluid processing device. The further away the magnet and the lower the strength of the magnetic field, the slower the magnetizing microparticles will pass through the multi-fluid density gradient column.

In an example, a maximum distance between the magnet and a nearest location where the first fluid layer resides along the multi-fluid density gradient column can be 50 mm, 40 mm maximum distance, 30 mm maximum distance, 20 mm maximum distance, or 10 mm maximum distance. The minimum distance, on the other hand, may be from 0.1 mm minimum distance, from 1 mm minimum distance, or 5 mm minimum distance. In one example, the minimum distance between the magnet and the multi-fluid density gradient column may be the thickness of the container or vessel that contains the multi-fluid density gradient column. Thus, distance ranges between the magnet and the multi-fluid density gradient column can be from 0.1 mm to 50 mm, from 1 mm to 50 mm, from 1 mm to 40 mm, from 1 mm to 30 mm, from 1 mm to 20 mm, from 1 mm to 10 mm, from 5 mm to 50 mm, or from 5 mm to 30 mm. In another example, a maximum distance between the magnet and a nearest location where the first fluid layer resides along the multi-fluid density gradient column can be 30 mm.

### Definitions

It is noted that, as used in this specification and the appended claims, the singular forms “a,” “an,” and “the” include plural referents unless the context clearly dictates otherwise.

As used herein, a plurality of items, structural elements, compositional elements, and/or materials may be presented in a common list for convenience. However, these lists should be construed as though members of the list are individually identified as separate and unique members. Thus, no individual member of such list should be construed as a de facto equivalent of any other member of the same list solely based on presentation in a common group without indications to the contrary.

Concentrations, amounts, and other numerical data may be expressed or presented herein in a range format. A range format is used merely for convenience and brevity and thus should be interpreted flexibly to include not only the numerical values explicitly recited as the limits of the range, but also to include individual numerical values or sub-ranges encompassed within that range as if numerical values and sub-ranges are explicitly recited. As an illustration, a numerical range of “1 wt % to 5 wt %” should be interpreted to include not only the explicitly recited values of 1 wt % to 5 wt %, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

The following illustrates an example of the present disclosure. However, the following is only illustrative of the application of the principles of the present disclosure. Numerous modifications and alternative compositions, methods, and systems may be devised without departing from the scope of the present disclosure. The appended claims are intended to cover such modifications and arrangements.

### EXAMPLE

Several fluid multi-density gradient columns were prepared that were used to introduce concentrated DNA into a master mix formulation of polymerase chain reaction (PCR) amplification. First, DNA was extracted from a 2.5×10⁵ live Streptococcus thermophilus bacteria culture using a multi-fluid density gradient column in accordance with the present disclosure. In further detail, eight different multi-fluid density gradient columns were prepared in 1.7 mL micro-centrifuge tubes with an opening in the bottom which was attached to a PCR reaction vessel. The top fluid layer (first fluid layer) included 300 μg DYNABEADS® DNA Direct Universal paramagnetic microparticles in 100 μL lysis buffer (from DYNABEADS® DNA Direct Universal Kit), which are commercially available from ThermoFisher Scientific (USA). The lower fluid layer (second fluid layer) of the multi-fluid density gradient columns included 0.5 g/mL sucrose in ultrapure H₂O.

Live Streptococcus thermophilus bacteria was added to the top fluid layer and allowed to incubate for 2 minutes, where the cells were chemically lysed and a portion of the released genomic DNA became bound to the DYNABEADS® DNA Direct Universal paramagnetic microparticles. Following the incubation period, a permanent rare earth magnet with 1 cm² surface area was placed beneath the multi-fluid density gradient column and the paramagnetic microparticles with DNA attached or attracted to the surfaces thereof were passed from the respective first fluid layer into the second fluid layer, and then ultimately into a PCR reaction vessel. The PCR reaction vessel included master mix (with was a third fluid) containing DNA polymerases, magnesium, dNTPs, primers, hydrolysis probes, bovine serum albumin, and buffer solution.

PCR can be carried out using Bio-Rad CFX96 Touch Real-Time PCR thermocycler. The PCR thermocycler in this instance is a fluid processing device, which can be part of a fluidic microchip, for example, that is pre-loaded with master mix. In this example, such as shown at 216 in FIG. 4, an egress opening can be sealed. Thus, the master mix may be densified as a third fluid with a third fluid density that is greater than the second fluid density, or the fluidic communication between the second fluid and the master mix may remain separate because of a minimal cross-sectional area at the fluid interface in combination with the fluid having no route of escape, e.g., no venting that might otherwise allow fluid flow to occur. Thus, the density gradient fluids may be loaded with highest density at the bottom, and once the magnetizing microparticles have been collected at the bottom of the fluid column, they may be actually inside of microfluidics chip and in contact with master mix. The microchip can then be used for thermocycling or some other processing. In this example, the thermocycler used may be considered to be a fluid processing device that includes electronic circuitry to operate the thermocycling process. The passing of the paramagnetic microparticles through the multi-fluid density gradient column did not significantly increase the PCR reaction times, as the paramagnetic microparticles moved quickly through the first and second layers of fluid.

While the present technology has been described with reference to certain examples, various modifications, changes, omissions, and substitutions can be made without departing from the spirit of the disclosure. The disclosure is to be limited only by the scope of the following claims.

What is claimed is:

1. A method of forming and loading a multi-fluid density gradient column, comprising:
forming a multi-fluid density gradient column by:
loading a first fluid having a first fluid density to form a first fluid layer, and
loading a second fluid having a second fluid density greater than the first fluid density to form a second fluid layer,
wherein the multi-fluid density gradient column is fluidly coupled to a fluid processing device; and
loading magnetizing microparticles that are surface-activated to bind with a biological component, or which are bound to the biological component, into the first fluid layer or the second fluid layer of the multi-fluid density gradient column.

2. The method of claim 1, wherein the second fluid is loaded from a bottom of the multi-fluid density gradient column to form the second fluid layer and the first fluid is loaded from a top of the multi-fluid density gradient column to form the first fluid layer.

3. The method of claim 1, wherein the first fluid and the second fluid are loaded sequentially from a bottom of the multi-fluid density gradient column to form the first fluid layer positioned on top of the second fluid layer.

4. The method of claim 1, further comprising loading a third fluid having a third fluid density in the multi-fluid density gradient column, and wherein the third fluid forms a third fluid layer based on the third fluid density in relation to the first fluid density of the first fluid and the second fluid density of the second fluid.

5. The method of claim 1, further comprising adjusting the first density of the first fluid, adjusting the second density of the second fluid, or adjusting both the first density of the first fluid and the second density of the second fluid prior to loading the first fluid and the second fluid into the multi-fluid density gradient column so that the second fluid density becomes greater than the first fluid density or so that a difference in the greater density of the second fluid density increases relative to the first fluid density.

6. The method of claim 1, wherein the multi-fluid density gradient column can include an inverted T-pipe associated with a valve, trapped gas, or a combination thereof to trap the second fluid in a channel extending upward from the inverted T-pipe.

7. A method of using a multi-fluid density gradient column in sample analysis, comprising:
loading a biological sample including a biological component and magnetizing microparticles that are surface-activated to bind with the biological component of the biological sample, or which are bound to the biological component of the biological sample, into a first fluid layer or a second fluid layer of a multi-fluid density gradient column, wherein the first fluid layer includes a first fluid having a first fluid density and the second fluid layer includes a second fluid having a second fluid density greater than the first fluid density;
exposing the magnetizing microparticles including the biological component bound thereto to a magnetic field to move the magnetizing microparticles including the biological component bound thereto from the first fluid layer into the second fluid layer;
passing the biological component to a fluid processing device through a fluidic outlet of the multi-fluid density gradient column; and
analyzing the biological component in the fluid processing device.

8. The method of claim 7, further comprising admixing the magnetizing microparticles and the biological sample in a loading solution before loading the biological sample and the magnetizing microparticles into the first fluid layer or the second fluid layer of the multi-fluid density gradient column.

9. The method of claim 7, wherein the passing of the biological component to the fluid processing device includes pumping the biological component into the fluid processing device via an injection pump, a syringe pump, a diaphragm pump, a peristaltic pump, or a combination thereof.

10. The method of claim 7, further comprising coating exposed surfaces on the magnetizing microparticles including the biological component bound thereto with a blocking agent prior to the analyzing of the biological component in the fluid processing device.

11. The method of claim 7, further comprising dissociating the biological component from the magnetizing microparticles prior to the analyzing of the biological component in the fluid processing device.

12. The method of claim 7, wherein the fluid processing device includes active circuitry including a sensor selected from a photo sensor, a thermal sensor, an optical sensor, a fluid flow sensor, a chemical sensor, an electrochemical sensor, a MEMS, or a combination thereof.

13. A microfluidic biological component concentration and processing system, comprising:
magnetizing microparticles that are surface-activated to bind with a biological component, or which are bound to the biological component;
a multi-fluid density gradient column to receive or containing the magnetizing microparticles, the multi-fluid density gradient column, including a first fluid layer having a first fluid density and a second fluid layer having a second fluid density that is greater than the first fluid density of the first fluid, wherein the second fluid layer is positioned vertically beneath the first fluid layer;
a magnet to draw the magnetizing microparticles from the first fluid layer into the second fluid layer;
a fluidic outlet fluidly coupled to the first fluid layer or the second fluid layer; and
a fluid processing device to receive modified fluid from the multi-fluid density gradient column, wherein the fluid processing device includes electronic circuitry that is interactive with the modified fluid.

14. The system of claim 13, wherein the fluid processing device includes a microfluidic chip including a microfluidic channel, wherein the microfluidic chip also includes active circuitry positioned to interact with the modified fluid, the biological component, or both within the microfluidic channel.

15. The system of claim 13, wherein the fluid processing system includes:
a first multi-fluid density gradient column associated with a first fluidic outlet that is fluidically connected a first fluid processing device; and
a second multi-fluid density gradient column associated with a second fluidic outlet that is fluidically connected to a second fluid processing device.
